(19) United States
(12) Patent Application Publication
(10) Pub. No.: US 2026/0191864 A1
(43) Pub. Date: Jul. 9, 2026

(54) Uses of Hydroimidazopyridopyrimidinone Derivatives for Managing Aneurysms or Other Vascular Conditions or Diseases

(71) Applicant: Emory University, Atlanta, GA (US)

(72) Inventors: Maria Alejandra San Martin, Atlanta, GA (US); Felipe Paredes-Diaz, Atlanta, GA (US)

(21) Appl. No.: 19/128,786

(22) PCT Filed: Nov. 9, 2023
(86) PCT No.: PCT/US2023/037103

§ 371 Date: May 9, 2025

Related U.S. Application Data
(60) Provisional application No. 63/424,153, filed on Nov. 10, 2022.

(51) Int. Cl. A61K 31/519; A61K 45/06; A61P 9/14

(52) U.S. Cl. CPC A61K 31/519; A61K 45/06; A61P 9/14

(57) ABSTRACT

This disclosure relates to methods of treating or preventing aneurysms or other vascular diseases of conditions related thereto comprising administering an effective amount of a hydroimidazopyridopyrimidinone derivative or salt thereof to a subject in need thereof. In certain embodiments, the derivative comprises a 2,4,6,7,8,9-hexahydro imidazo[1,2-a]pyrido[3,4-e]pyrimidin-5 (1H)-one core structure comprising substituents. In certain embodiments, the compound is 7-benzyl-4-(2-methylbenzyl)-2,4,6,7,8,9-hexa-hydroimidazo[1,2-a]pyrido[3,4-e]pyrimidin-5(1H)-one (ONC201/TIC10), derivative, or salt thereof.

### CROSS-REFERENCE TO RELATED APPLICATIONS

This application claims the benefit of U.S. Provisional Application No. 63/424,153 filed Nov. 10, 2022. The entirety of this application is hereby incorporated by reference for all purposes.

### BACKGROUND

An aneurysm is a condition in which a blood vessel abnormally expands or bulges and sometimes ruptures. Aneurysms are often caused by progressive weakening of a blood vessel wall near branch points, and commonly occur in certain locations such as in the aorta, brain, neck, groin, or behind the knees. Brain aneurysms may result in loss of consciousness, vasospasms, hydrocephalus (buildup of brain fluid), ischemic stroke, and death. Aortic aneurysms create a bulge in the large artery that directs blood from the heart through the chest and are often subcategorized as thoracic or abdominal. Certain aneurysms are treated by surgical interventions. Without surgery, the annual survival rate of an aortic aneurysm is of 20%. The risk of rupture of the abdominal aortic aneurysm increases with size, wherein aneurysms larger than 6 cm have a 25% annual risk of rupture. Following the rupture of an abdominal aortic aneurysm, the risk of death is approximately 80%. Most patients die before reaching the hospital. Therapeutic treatments are typically limited to relieving symptoms and managing complications. Blood pressure medications, cholesterol lowering drugs, and anti-clotting agents may be prescribed; however, these strategies are not universally effective. Thus, there is a need to identify improvements.

Kim et al. report that there is insufficient evidence to recommend beta-blocker therapy for the purpose of abdominal aortic aneurysm sac regression. J Vasc Surg, 2017, 65:337-45.

Connolly et al. report guidelines for the management of aneurysmal subarachnoid hemorrhage. Stroke, 2012, 43:1711-1737.

Pruss et al. report dual metabolic reprogramming by ONC201/TIC10 and 2-deoxyglucose induces energy depletion and synergistic anti-cancer activity in glioblastoma. British J Cancer, 2020, 122:1146-1157.

References cited herein are not an admission of prior art.

### SUMMARY

This disclosure relates to methods of treating or preventing aneurysms or other vascular diseases or conditions related thereto comprising administering an effective amount of a hydroimidazopyridopyrimidinone derivative or salt thereof to a subject in need thereof. In certain embodiments, the derivative comprises a 2,4,6,7,8,9-hexahydroimidazo[1,2-a]pyrido[3,4-e]pyrimidin-5 (1H)-one core structure comprising substituents. In certain embodiments, the compound is 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative or salt thereof.

In certain embodiments, this disclosure relates to methods of treating or preventing an aneurysm, aneurysmal subarachnoid hemorrhage, or hemorrhagic stroke comprising administering an effective amount of 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof to a subject in need thereof.

In certain embodiments, this disclosure relates to methods of inducing vascular smooth muscle differentiation in vivo comprising administering an effective amount of 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof to a subject in need thereof.

In certain embodiments, this disclosure relates to methods of treating or preventing a cardiovascular disease such as atherosclerosis comprising administering an effective amount of 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof to a subject in need thereof.

In certain embodiments, 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof is administered in combination with a blood pressure lowing medication, beta-blocker, statin, anti-inflammatory agent, cholesterol lowering drug, clotting agent, an anti-blood clotting agent, ACE inhibitor, or combinations thereof.

In certain embodiments, this disclosure relates to pharmaceutical compositions comprising 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof and a pharmaceutically acceptable excipient. In certain embodiments, this disclosure relates to uses of pharmaceutical compositions comprising 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof in the preparation of a medicament for treating or preventing an aneurysm, aneurysmal subarachnoid hemorrhage, or hemorrhagic stroke.

### BRIEF DESCRIPTION OF THE SEVERAL VIEWS OF THE DRAWINGS

FIGS. 1A-1D show data indicating the drug Onc201 (TIC10) induces vascular smooth muscle differentiation in vivo and inhibits the formation of aortic aneurysms in mice. It is contemplated that VSMC phenotypic modulation is responsible for foam cell formation during atherosclerosis.

FIG. 1A shows data on the diameter of the aorta on day 14.

FIG. 1B shows data on echocardiogram quantified differences in aortic diameters between baseline and at 14 days. Data represents percentage of maximal aortic dilation on day 14 of each animal. The recording and subsequent analysis were blinded to the assignment of the treatment regimen.

FIG. 1C shows data where elastin fibers were stained with Verhoeff-Van Gieson (VVG) staining. Images were quantified using ImageJ plus/color deconvolution plugin.

FIG. 1D shows quantitative data on ACTA2 (smooth muscle actin) staining using a specific antibody (A2547, Sigma).

### DETAILED DISCUSSION

Before the present disclosure is described in greater detail, it is to be understood that this disclosure is not limited to embodiments as described, and as such may, of course, vary. It is also to be understood that the terminology used herein is for the purpose of describing embodiments, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

A “hemorrhagic stroke” refers to a bleeding event caused by a ruptured artery in the brain. Rupture of a brain aneurysm is one of the main causes. Individuals with an abnormal blood vessel formation, i.e., arteriovenous malformation (AVM), are at higher risk to develop aneurysms that can then burst due to high blood pressure and/or due to a blood vessel wall becoming weak because of a bulging of the aneurysm. The hole in the artery wall causes blood to spill into the tissue surrounding the artery. A subarachnoid hemorrhage refers to a ruptured brain aneurysm that occurs in the space between the brain and the thin tissues covering the brain.

The terms “ONC201” or “TIC10” refer to the compound with the chemical name, 7-benzyl-4-(2-methylbenzyl)-2,4,6,7,8,9-hexahydroimidazo[1,2-a]pyrido[3,4-e]pyrimidin-5 (1H)-one, as reported in Wagner et al., The angular structure of ONC201, a TRAIL pathway-inducing compound, determines its potent anti-cancer activity, Oncotarget, 2014, 5 (24): 12728-12737, and as illustrated with the chemical structure provided below.

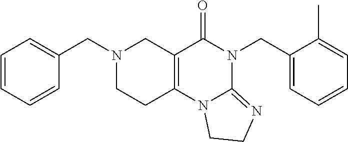 [SMILES: Cc1ccccc1CN1C(=O)C2=C(CCN(Cc3ccccc3)C2)N2CCN=C12]

The present chemical entities, pharmaceutical compositions and methods are meant to include all such possible isomers, including racemic mixtures, tautomer forms, hydrated forms, optically substantially pure forms, and intermediate mixtures. Unless otherwise stated, structures depicted herein are also meant to include compounds which differ only in the presence of one or more isotopically enriched atoms. For example, compounds having the present structures except for the replacement or enrichment of a hydrogen by deuterium or tritium at one or more atoms in the molecule, or the replacement or enrichment of a carbon by ¹³C or ¹⁴C at one or more atoms in the molecule, are within the scope of this disclosure. In some embodiments, provided herein are compounds that can also contain unnatural proportions of atomic isotopes at one or more of atoms that constitute such compounds. All isotopic variations of the compounds as disclosed herein are encompassed within the scope of the present disclosure.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters or enol ethers. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide and benzamide derivatives of an amine functional group in the active compound and the like.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom, or replacing an amino group with a hydroxy group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(—O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen, hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral salts such as sodium, potassium, or zinc carboxylic acid salts, or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

### Hydroimidazopyridopyrimidinone Derivatives

In certain embodiments, this disclosure relates to hydroimidazopyridopyrimidinone derivatives or salts thereof. In certain embodiments, the hydroimidazopyridopyrimidinone derivative is the compound 7-benzyl-4-(2-methylbenzyl)-2,4,6,7,8,9-hexahydroimidazo[1,2-a]pyrido[3,4-e]pyrimidin-5 (1H)-one or salt thereof. In certain embodiments, the derivative has the following formula,

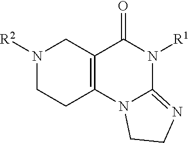

or salts thereof wherein,
R¹ is benzyl optionally substituted with one or more, the same or different, R¹⁰,
R² is benzyl optionally substituted with one or more, the same or different, R¹⁰,
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹⁰ is optionally substituted with one or more, the same or different, R¹¹, and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

### Methods of Use

This disclosure relates to methods of treating or preventing aneurysms or conditions related thereto comprising administering an effective amount of a hydroimidazopyridopyrimidinone derivative or salt thereof to a subject in need thereof. In certain embodiments, this disclosure relates to methods of treating or preventing an aneurysm comprising administering an effective amount of 7-benzyl-4-(2-methylbenzyl)-2,4,6,7,8,9-hexahydroimidazo[1,2-a]pyrido[3,4-e]pyrimidin-5 (1H)-one (ONC201/TIC10) or salt thereof to a subject in need thereof. In certain embodiments, the subject is a human patient.

In certain embodiments, the subject is at risk or, exhibiting symptoms of, or diagnosed with an aneurysm, atherosclerosis, or other vascular disease or condition. In certain embodiments, the subject is diagnosed with an aortic aneurysm or brain aneurysm, neck aneurysm, femoral arterial aneurysm, a pulsatile mass in the groin, or popliteal artery aneurysm, or aneurysm in the wall of the artery located behind the knee joint. In certain embodiments, the subject exhibits symptoms of increase blood pressure or disruption of the blood/oxygen supply, loss of consciousness, vasospasms, hydrocephalus (buildup of brain fluid), and/or ischemic stroke.

In certain embodiments, this disclosure relates to methods of treating or preventing aneurysmal subarachnoid hemorrhage comprising administering an effective amount of 7-benzyl-4-(2-methylbenzyl)-2,4,6,7,8,9-hexahydroimidazo[1,2-a]pyrido[3,4-e]pyrimidin-5 (1H)-one or salt thereof to a subject in need thereof. In certain embodiments, the subject is a human patient.

In certain embodiments, the subject is at risk or, exhibiting symptoms of, or diagnosed with an aneurysm. In certain embodiments, the 7-benzyl-4-(2-methylbenzyl)-2,4,6,7,8,9-hexahydroimidazo[1,2-a]pyrido[3,4-e]pyrimidin-5 (1H)-one or salt thereof is administered in combination with another medicament. In certain embodiments, the medicament is antihypertensive medication or cholesterol lowing medication.

In certain embodiments, the subject is at risk of an aneurysm due to having high blood pressure (e.g., systolic above 120, 130, or 140 mm Hg, and/or diastolic above 80 or 90 mm Hg), or systolic above 140 mm Hg and diastolic above 90 mm Hg.

In certain embodiments, the subject is at risk of an aneurysm due to having high cholesterol at or above 240 mg/dL in the blood.

In certain embodiments, the subject is at risk of an aneurysm due to having atherosclerosis (hardened arteries due to plaque).

In certain embodiments, the subject is at risk of an aneurysm due to being over 50, 55 or 65 years of age and optionally having other risk factors or conditions reported herein.

In certain embodiments, the subject is at risk of an aneurysm due to being chronic cigarette smoker.

In certain embodiments, the subject is at risk of an aneurysm due to chronic use of drugs that create high blood pressure. In certain embodiments, the drug is alcohol or cocaine. In certain embodiments, the subject consumes more than 200 or 300 grams of alcohol per week.

In certain embodiments, the subject is at risk of an aneurysm due to a head injury.

In certain embodiments, the subject is at risk of an aneurysm due to a bacterial or viral infection in the blood.

In certain embodiments, the subject is diagnosed with Marfan syndrome, Loeys-Dietz syndrome, or Ehlers-Danlos syndrome.

In certain embodiments, the subject is at risk of an aneurysm due to polycystic kidney disease.

In certain embodiments, the subject is at risk of an aneurysm due to a narrow aorta (coarctation of the aorta).

In certain embodiments, the subject is at risk of an aneurysm due to a brain arteriovenous malformation (AVM).

In certain embodiments, the subject is at risk of an aneurysm due to family history of brain aneurysm, particularly a first-degree relative, such as a parent, brother, sister, or child.

In certain embodiments, the aneurysm is in a heart such as a thoracic aorta or abdominal aorta.

In certain embodiments, the aneurysm is in a brain (cerebral aneurysm).

In certain embodiments, the aneurysm is behind the knee in a leg (popliteal artery aneurysm).

In certain embodiments, the aneurysm is in an intestine (mesenteric artery aneurysm).

In certain embodiments, the aneurysm is in a spleen (splenic artery aneurysm).

In certain embodiments, the subject is less than 6 months old and/or the aneurysms was present at birth (congenital).

In certain embodiments, the subject is diagnosed with a defect in an artery wall.

In certain embodiments, the subject is at risk of developing an aneurysm due to high blood pressure, high cholesterol, cigarette smoking or combinations thereof.

In certain embodiments the subject has atherosclerosis (cholesterol buildup in arteries).

In certain embodiments the subject has fibromuscular dysplasia.

In certain embodiments the subject is pregnant and optionally at risk of formation and rupture of splenic artery aneurysms.

In certain embodiments, this disclosure relates to the use of an effective amount of 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof in the production of a medicament for uses in treating or preventing aneurysms as reported herein. In certain embodiments, the subject is a human patient.

In certain embodiments, 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof is administered in combination with a blood pressure lowing medications, beta-blocker, angiotensin-converting enzyme (ACE) inhibitor, calcium channel blocker, statin, anti-inflammatory agent, cholesterol lowering drug, an anticoagulant, or combinations thereof.

In certain embodiments, the medicament is a beta blocker such as acebutolol, atenolol, betaxolol, bisoprolol, carteolol, metoprolol, nadolol, penbutolol, pindolol, propranolol, timolol, or salts thereof.

In certain embodiments, the medicament is a pain reliever such as acetaminophen, naproxen, ibuprofen, aspirin, codeine, fentanyl, hydrocodone, hydromorphone, meperidine, morphine, oxycodone, tramadol, or salts thereof.

In certain embodiments, the medicament is an angiotensin-converting enzyme (ACE) inhibitor such as benazepril, captopril, enalapril, fosinopril, lisinopril, moexipril, perindopril, quinapril, ramipril, trandolapril, or salts thereof.

In certain embodiments, the medicament is a calcium channel blocker such as nimodipine, amlodipine, diltiazem, felodipine, isradipine, nicardipine, nifedipine, nisoldipine, verapamil, or salts thereof.

In certain embodiments, the medicament is an anti-seizure medication such as levetiracetam, phenytoin, or valproic acid or salts thereof for use in the context of a brain aneurysm.

In certain embodiments, the medicament is an angiotensin II receptor blocker such as losartan, valsartan, olmesartan or salts thereof.

In certain embodiments, the medicament is a lipid lower agent such as a statin, ezetimibe, cholestyramine, colestipol, colesevelam, PCSK9 inhibitors such as alirocumab and evolocumab, or salts thereof.

In certain embodiments, the medicament is a statin such as atorvastatin, fluvastatin, lovastatin, pravastatin, rosuvastatin, simvastatin, or salts thereof.

In certain embodiments, the medicament is anticoagulant such as heparin, warfarin, rivaroxaban, dabigatran, apixaban, edoxaban, clopidogrel, dipyridamole, ticlopidine, or salts thereof.

In certain embodiments, this disclosure relates to methods of treating or preventing vascular diseases comprising administering an effective amount of 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof to a subject in need thereof.

The precise therapeutically effective amount of the compound(s) of this disclosure will depend on a number of factors. There are variables inherent to the compounds including, but not limited to, the following: molecular weight, absorption, bioavailability, distribution in the body, tissue penetration, half-life, metabolism, protein binding, and excretion. These variables determine what dose of compound needs to be administered in a sufficient percentage and for a sufficient amount of time to have the desired effect on the condition being treated. The duration of drug exposure will be limited only by the compound half-life, and side effects from treatment requiring cessation of dosing. The amount of compound administered will also depend on factors related to patients and disease including, but not limited to, the following: the age, weight, concomitant medications, and medical condition of the subject being treated, the precise condition requiring treatment and its severity, the nature of the formulation, and the route of administration. Ultimately the dose will be at the discretion of the attendant physician or veterinarian. Typically, the compound disclosed herein will be given for treatment in the range of 0.01 to 30 mg/kg body weight of recipient (human patient) per day or per dose or per cycle of treatment and more usually in the range of 0.1 to 10 mg/kg body weight per day or per dose or per cycle of treatment. Thus, for an adult human being treated for a condition, the actual amount per day or per dose or per cycle of treatment would usually be from 1 to 2000 mg and this amount may be given in a single or multiple doses per day or per dose or per cycle of treatment. Dosing regimens may vary significantly and will be determined and altered based on clinical experience with the compound. The full spectrum of dosing regimens may be employed ranging from continuous dosing (with daily doses) to intermittent dosing. A therapeutically effective amount of a pharmaceutically acceptable salt of a compound disclosed herein may be determined as a proportion of the therapeutically effective amount of the compound as the free base.

### Pharmaceutical Compositions

In certain embodiments, this disclosure relates to pharmaceutical compositions comprising 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof and a pharmaceutically acceptable excipient.

In certain embodiments, the pharmaceutically acceptable excipient is selected from lactose, sucrose, mannitol, glucose, galactose, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition in the form of a table, pill, capsule, or gel. In certain embodiments, the pharmaceutical composition in the form or a liquid comprising pH buffering agents and optionally salts and/or saccharide or polysaccharide. In certain embodiments, this disclosure contemplates an intravenous formulation with pH buffering agents and tonicity in a range representing physiological values (pH 7 to 8) or for bolus administration, e.g., containing normal saline or dextrose optionally containing pH buffering agents. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutical composition is in the form of a tablet, pill, capsule, powders, granules, gel, gel capsule, or cream. In such solid dosage forms, the active compound is admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia gum, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof.

While it is possible that, for use in therapy, a therapeutically effective amount of a compound disclosed herein may be administered as the raw chemical, it is typically presented as the active ingredient of a pharmaceutical composition or formulation. Accordingly, the disclosure further provides a pharmaceutical composition comprising a compound disclosed herein. The pharmaceutical composition may further comprise one or more active agents or pharmaceutically acceptable carriers, diluents, and/or excipients. The active agent may be contained in the pharmaceutical composition in combination with any of the aforementioned drugs such as a blood pressure lowing agent, beta-blocker, angiotensin-converting enzyme (ACE) inhibitor, calcium channel blocker, statin, anti-inflammatory agent, cholesterol lowering agent, an anticoagulant, or combinations thereof.

The carrier(s), diluent(s) and/or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing a compound disclosed herein with one or more pharmaceutically acceptable carriers, diluents and/or excipients to provide a medicinal product.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound disclosed herein (as a free-base, solvate (including hydrate) or salt, in any form), depending on the condition being treated, the route of administration, and the age, weight and condition of the patient. Preferred unit dosage formulations are those containing a daily dose, weekly dose, or monthly dose, of an active ingredient. Furthermore, such pharmaceutical formulations may be prepared by any of the methods well known in the pharmacy art.

Pharmaceutical formulations may be adapted for administration by any appropriate route, for example by the oral (including capsules, tablets, liquid-filled capsules, disintegrating tablets, immediate, delayed and controlled release tablets, oral strips, solutions, syrups, buccal and sublingual), rectal, nasal, inhalation, topical (including transdermal), vaginal or parenteral (including subcutaneous, intramuscular, intravenous or intradermal) route. Such formulations may be prepared by any method known in the art of pharmacy, for example by bringing into association the active ingredient with the carrier(s), excipient(s) or diluent. Generally, the carrier, excipient or diluent employed in the pharmaceutical formulation is “non-toxic,” meaning that it/they is/are deemed safe for consumption in the amount delivered in the pharmaceutical composition, and “inert” meaning that it/they does/do not appreciably react with or result in an undesired effect on the therapeutic activity of the active ingredient.

Pharmaceutical formulations adapted for oral administration may be presented as discrete units such as liquid-filled or solid capsules; immediate, delayed, or controlled release tablets; powders or granules; solutions or suspensions in aqueous or non-aqueous liquids; edible foams or whips; oil-in-water liquid emulsions, water-in-oil liquid emulsions or oral strips, such as impregnated gel strips.

For instance, for oral administration in the form of a tablet or capsule, the active drug component can be combined with an oral pharmaceutically acceptable carrier such as ethanol, glycerol, sterilized water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Solid capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating, slugging, and/or adding a lubricant and disintegrant and pressing into tablets. A powder mixture may be prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compounds disclosed herein can also be combined with a free-flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Solutions and syrups can be prepared by dissolving the compound in a suitably flavored aqueous solution, while elixirs are prepared through the use of a pharmaceutically acceptable alcoholic vehicle. Suspensions can be formulated by dispersing the compound in a pharmaceutically acceptable vehicle. Solubilizers and emulsifiers such as ethoxylated isostearyl alcohols and polyoxyethylene sorbitol ethers, preservatives, flavor additive such as peppermint oil or natural sweeteners or saccharin or other artificial sweeteners, and the like can also be added.

Where appropriate, unit dosage formulations for oral administration can be microencapsulated. The formulation can also be prepared to prolong or sustain the release as for example by coating or embedding particulate material in polymers, wax, or the like.

The compounds of the disclosure can also be administered in the form of liposome delivery systems, such as small unilamellar vesicles, large unilamellar vesicles and multilamellar vesicles. Liposomes can be formed from a variety of phospholipids, such as cholesterol, stearyl amine, or phosphatidylcholines.

Pharmaceutical formulations adapted for topical administration in the mouth include lozenges, pastilles, and mouth washes. Suitable formulations wherein the carrier is a liquid, for administration as a nasal spray or as nasal drops, include aqueous or oil solutions of the active ingredient. Pharmaceutical formulations adapted for administration by inhalation include fine particle dusts or mists, which may be generated by means of various types of metered dose pressurized aerosols, metered dose inhalers, dry powder inhalers, nebulizers, or insufflators.

Pharmaceutical formulations adapted for parenteral administration include aqueous and non-aqueous sterile injection solutions which may contain anti-oxidants, buffers, bacteriostats and solutes which render the formulation of pharmaceutically acceptable tonicity with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules, and tablets.

In certain embodiments, this disclosure contemplates kits comprising 7-benzyl-4-(2-methylbenzyl)-2,4,6,7,8,9-hexahydroimidazo[1,2-a]pyrido[3,4-e]pyrimidin-5 (1H)-one (ONC201/TIC10), derivative, or salt thereof as disclosed herein and optionally a container, and optionally a pharmaceutically acceptable excipient, and optionally another agent such as blood pressure lowing medications, beta-blocker, statin, anti-inflammatory agent, cholesterol lowering drug, clotting agent, an anti-blood clotting agent, ACE inhibitor or combinations thereof. The container may contain be marked with a label or contain a pamphlet with instructions for use. In certain embodiments, the container is a vial, capsule, syringe, or bottle/vial adapted with a septum for drawing liquid contents with a needle, syringe, canula, or other transfer device.

### Examples

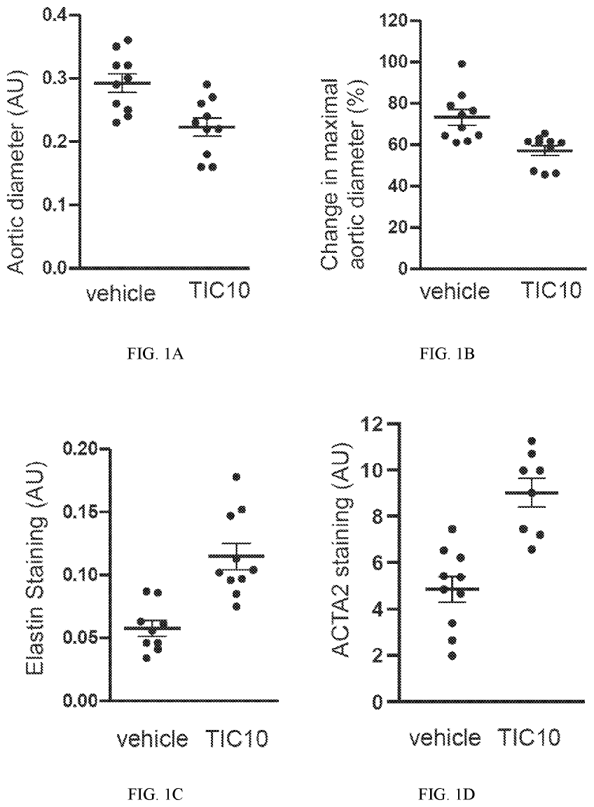

Data indicates that the drug ONC201 (TIC10) induces vascular smooth muscle differentiation in vivo and inhibits the formation of aortic aneurysms in mice (FIGS. 1A-D). This drug is currently in clinical trials for cancer treatment, but its action has not been linked to muscle or cardiovascular diseases.

What is claimed is:

1. A method of treating or preventing an aneurysm comprising administering an effective amount of 7-benzyl-4-(2-methylbenzyl)-2,4,6,7,8,9-hexahydroimidazo[1,2-a]pyrido[3,4-e]pyrimidin-5 (1H)-one or salt thereof to a subject in need thereof.

2. The method of claim 1, wherein the subject is at risk or, exhibiting symptoms of, or diagnosed with an aneurysm.

3. The method of claim 1, wherein the subject is at risk of an aneurysm due to having high blood pressure.

4. The method of claim 1, wherein the subject is at risk of an aneurysm due to having high cholesterol.

5. The method of claim 1, wherein the subject is at risk of an aneurysm due to having atherosclerosis.

6. The method of claim 1, wherein the subject is at risk of an aneurysm due to being over 55 years of age.

7. The method of claim 1, wherein the subject is at risk of an aneurysm due to being chronic cigarette smoker.

8. The method of claim 1, wherein the subject is at risk of an aneurysm due to chronic use of drugs that create high blood pressure.

9. The method of claim 1, wherein the subject is at risk of an aneurysm due to a head injury.

10. The method of claim 1, wherein the subject is diagnosed with Marfan syndrome, Loeys-Dietz syndrome, or Ehlers-Danlos syndrome.

11. The method of claim 1, wherein the subject is at risk of an aneurysm due to a brain arteriovenous malformation (AVM).

12. The method of claim 1 wherein 7-benzyl-4-(2-methylbenzyl)-2,4,6,7,8,9-hexahydroimidazo[1,2-a]pyrido[3,4-e]pyrimidin-5 (1H)-one or salt thereof is administered in combination with another medicament.

13. A method of treating or preventing hemorrhagic stroke comprising administering an effective amount of 7-benzyl-4-(2-methylbenzyl)-2,4,6,7,8,9-hexahydroimidazo[1,2-a]pyrido[3,4-e]pyrimidin-5 (1H)-one or salt thereof to a subject in need thereof.

14. A method of treating or preventing aneurysmal subarachnoid hemorrhage comprising administering an effective amount of 7-benzyl-4-(2-methylbenzyl)-2,4,6,7,8,9-hexahydroimidazo[1,2-a]pyrido[3,4-e]pyrimidin-5 (1H)-one or salt thereof to a subject in need thereof.

15-18. (canceled)
